(19) United States
(12) Patent Application Publication
(10) Pub. No.: US 2005/0158376 A1
(43) Pub. Date: Jul. 21, 2005

(54) Dietary supplement and method of processing same

(76) Inventors: William F. Sardi, La Verne, CA (US); Stanley H. Solomonson, Encinitas, CA (US)

Correspondence Address: EDELL, SHAPIRO & FINNAN, LLC, 1901 RESEARCH BOULEVARD, SUITE 400, ROCKVILLE, MD, 20850, US

(21) Appl. No.: 10/971,017

(22) Filed: Oct. 25, 2004

Related U.S. Application Data
(60) Provisional application No. 60/513,225, filed on Oct. 23, 2003.

(51) Int. Cl. A61K 9/48; A61K 31/05

(52) U.S. Cl. 424/451; 514/734

(57) ABSTRACT

A composition including small plant or synthetic molecules, such as resveratrol and quercetin, in their biologically active form, is provided. The small molecules may be derived from natural sources such as grapes or Giant Knotweed (botanical name Polygonum). The composition may be processed such that the gene-controlled biological activity of the small molecules is maintained. Specifically, the raw material for encapsulation must exhibit biological activity before and after encapsulation and be processed in an oxygen-free (nitrogen), dim light environment. The composition may further include at least one metal chelating agent as an antioxidant stabilizer, an antioxidant, and an emulsifier. The compositions are useful in the formation of a dietary supplement or drug.

### CROSS REFERENCE TO RELATED APPLICATIONS

This application claims priority from U.S. Provisional Patent Application Ser. No. 60/513,225, entitled “Dietary Supplement and Method of Processing Same” and filed Oct. 23, 2003. The disclosure of the above-mentioned provisional application is incorporated herein by reference in its entirety.

### FIELD OF THE INVENTION

The present invention relates to an encapsulated composition including biologically active small molecules and a method of processing the composition to maintain biological activity and, in particular, to maintain its ability to activate (express) or deactivate (silence) the Sirtuin 1 gene.

### BACKGROUND

Resveratrol is a naturally occurring phenolic fungicide produced by some plants in response to injury or fungal infection. It is one of a group of compounds (called phytoalexins) produced in plants during times of environmental stress such as adverse weather or attacks by insects, animals, or pathogenic microorganisms (e.g., fungi). Resveratrol is the parent molecule of a family of polymers called viniferins. Its natural sources include pine trees, eucalyptus plants, knotweed, Liliaceae, Polygonaceae, Leguminosae, mulberries, peanuts, blueberries, cranberries, and pine-kernels. Its most abundant natural sources are Vitis vinifera, Vitis labrusca, and Vitis rotundifolia (muscadine), all of which are grape varieties used to make wine.

Resveratrol (C₁₄H₁₂O₃), also known as 3,4′,5 trihydroxystilbene, naturally exists in cis- and trans-stereoisomeric forms. In grapes and wine, resveratrol is present mainly in its trans stereoisomeric form. During the manufacturing process of wine, fermentation takes place while the seeds and skins of the grapes are left in contact with the pressed juice. Ethanol, created during the fermentation process, naturally extracts and concentrates resveratrol from the skins and seeds. The resveratrol content of a wine depends not only on the type of grape, but also on the length of time the grape skins/seeds are present during the fermentation process (i.e., the amount of time the skins/seeds are left in contact with the juice). The resveratrol concentration is significantly (approximately ten times) higher in red wine than in white wine because during red wine production, the skins/seeds are left in contact with the juice for a longer period of time, increasing the amount of resveratrol from the skins/seeds. In a typical (750 ml) bottle of red wine, the total resveratrol concentration may range from 0.6 to 15 mg/l. Once formed, the resveratrol is preserved within the wine by its via its packaging and processing, namely, it is stored in an airtight bottle substantially impervious to light. The bottles themselves, moreover, are typically stored in cool, darkened environments (e.g., a wine cellar).

Studies have shown that resveratrol is biologically active, providing several health benefits including cancer prevention, anti-inflammatory properties, and cardiovascular effects. Resveratrol functions as a moderate antioxidant, quenching free radical damage linked to several cancers. Resveratrol also inhibits the transcription factor NF-κB, which stimulates genes responsible for cell survival, inflammation, and proliferation of cancer. When applied to cancer cells, resveratrol sensitizes them to tumor necrosis factor-α, which initiates apoptosis (cell death). Epidemiological, in vitro, and animal studies also suggest that a high resveratrol intake is associated with a reduced incidence of cardiovascular disease (producing antiplatelet and hypolipidemic effects). More recent studies suggest resveratrol increases the life span of yeast, worms, and mice by slowing cellular degeneration. It has been suggested that 3 to 15 milligrams of resveratrol (the amount present in 3-5 glasses of red wine) is believed to be sufficient to produce the above effects.

In order to provide the aforementioned benefits, however, the resveratrol, once produced, must remain biologically active. Maintaining the biological activity of resveratrol is difficult. Resveratrol has a half-life of about one day; consequently, upon exposure to the ambient environment, it completely oxidizes within two days. Once oxidized, its ability to affect biological systems diminishes.

When used in dietary supplements, raw resveratrol is generally produced as an alcohol extract from plant sources (e.g., Giant Knotweed), which is then dried into a powder, encapsulated or put into pill form, which, in turn, are sealed in airtight packaging. Although resveratrol as a raw material generally exhibits effective biological activity, when the resveratrol powder is mixed and blended in the process of encapsulation, it is exposed to oxygen, eventually losing some if not all of its biological activity before it can reach the consumer. As a result, even though the resveratrol molecule may be confirmed to be present after encapsulation by high performance liquid chromatography (HPLC), it may or may not exhibit gene-controlled biological activity. Consequently, even though resveratrol may be present in the supplement, it may lack significant biological activity. In particular, while resveratrol may exhibit certain antioxidant, estrogen-like, cholesterol-controlling effects, it loses its genomic biological activity. Enzymatic biological activity (e.g., the ability to activate and deactivate human enzymes) has been demonstrated only in preserved resveratrol molecules (e.g., as the molecules exist is in wine or in research-grade resveratrol). Consequently, it is desirable to provide a dietary supplement including stabilized resveratrol that retains its biological activity and, in particular, its enzymatic biological activity.

### OBJECTS AND SUMMARY OF THE INVENTION

Therefore, in light of the above, and for other reasons that become apparent when the invention is described, an object of the present invention is to provide an encapsulated composition including a biologically active resveratrol, and to a method of encapsulating the composition such that the resveratrol remains biologically active when ingested by a consumer.

It is another object of the invention to provide a method of encapsulating small molecule plant polyphenols capable of stimulating enzymatic activity such that the enzymatic activity is preserved.

It is a further object of the invention to provide an encapsulated composition having a genomic effect, including activation and/or deactivation of human enzymes.

It is yet another object of the invention to provide a dietary supplement including an encapsulated resveratrol composition in which the efficacy of the resveratrol is maintained by preventing its exposure to oxygen during the encapsulation process.

A still further object of the present invention is to prevent metal-induced oxidation of dietary supplement ingredients during the processing and packaging of the supplement.

Generally, the embodiments of the present invention provide an encapsulated composition including (1) small molecules of a plant or synthetic source in their biologically active form and (2) optionally at least one of (a) an emulsifier, (b) an antioxidant, and (c) a chelating agent. The embodiments further provide a method of encapsulating a composition including the steps of (1) deriving material including biologically active small molecules derived from a plant source or formed synthetically and (2) encapsulating the material in a substantially oxygen-free environment.

The above and still further objects, features and advantages of the present invention will become apparent upon consideration of the following detailed description of specific embodiments thereof.

### DETAILED DESCRIPTION

One embodiment of the invention relates to a composition including small molecules derived from a plant or formed synthetically, and a method of encapsulating the composition such that it maintains its biological activity for extended periods of time. The biologically active small molecules of plant or synthetic source may include small molecule plant polyphenols such as resveratrol, quercetin, fisetin, butein, piceatonol, isoliquiritigenin, which, by virtue of their small size and molecular weight, are able to pass through cell walls, enter the cell nucleus, and alter certain gene-controlled mechanisms. A preferred small molecule plant polyphenol is resveratrol (molecular weight 228.25), which has been shown to activate Sirtuin-1 controlled enzyme activity better than other polyphenols. However, other small molecules may be employed. Resveratrol may be formed synthetically or derived naturally from sources such as plant material, including grapes and knotweed. Preferably, the resveratrol includes the trans stereoisomer of the molecule, namely, trans-3,4′,5 trihydoxystilbene, which possesses the following chemical structure: 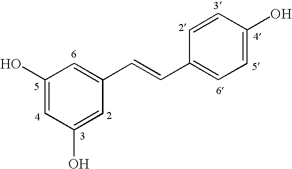
To maintain biological activity for an “extended period” of time, the small molecules of plant or synthetic source preferably remain biologically active for time periods after which the molecules would naturally become biologically inactive due to degradative processes such as oxidation. For example, resveratrol possesses a half-life of approximately one day; consequently, it typically loses significant biological activity within two days of exposure to ambient conditions and during processing of dietary supplements.

Another embodiment of the invention relates to a method of encapsulating small molecules of plant or synthetic source including the steps of (1) deriving material including small molecules of plant or synthetic source; (2) encapsulating the material in a substantially oxygen-free environment; and (3) optionally adding (a) a chelating agent, (b) an antioxidant, and/or (c) an emulsifier to the material.

As discussed above, the material including small molecules may be derived naturally or synthetically formed. The material, moreover, should be biologically active. Biological activity is intended to include the ability of the small molecules to pass through a cell wall, enter the cell nucleus, and beneficially alter gene-controlled enzymes, in particular, the Sirtuin 1 enzyme. Preferably, the biologically active material is naturally derived, i.e., derived from at least one natural source such as plants (or parts thereof, such as tubers or fruit (including pulp and skins) from the plant). One preferred source is the seeds and/or skins of grapes, such as Vitis vinifera, Vitis labrusca, and Vitis rotundifolia. Another preferred source is Polygonum (Giant Knotweed) and, in particular, 1-polygonum cuspidatum (a species of giant knotweed). The natural derivation process includes those processes generally known in the art, including an extraction process in which a solvent is used to extract the small molecules from a natural source. The solvent includes aqueous solvents, organic solvents, and mixtures thereof. The solvent may include, but is not limited to, alcohols such as ethanol. By way of specific examples, the extracted small molecule material may include aqueous or organic solvent extracts of plants (or parts thereof), fruit juices (e.g., grape juice), and fermented liquors (e.g. wine) produced from plants or fruit juice, or mixtures of any of the foregoing. The extracted material may further include inert plant material naturally removed during the extraction process. The extracted material may be processed (physically and/or chemically) to remove the solvent and increase the concentration of the small molecules. For example, the solvent may be removed from the extract (e.g., by drying), leaving a dried powder.

The derived material including small molecules is then encapsulated in a substantially oxygen-free environment. As used herein, the phrase “substantially oxygen-free” is intended to include environments having less than less than about 100 parts per million oxygen. Ideally, the encapsulation process would take place immediately after the extraction or formation of the small molecules and be shielded from exposure to light, heat, and oxygen. Alternatively, the material including small molecules may be stored in a substantially oxygen-free environment until encapsulated.

The encapsulation process includes the steps of (1) providing a capsule including a head portion and a body portion; (2) at least partially filling the body portion with the material including biologically active small molecules; (3) axially positioning the head portion over the body portion such that the portions at least partially overlap; and (4) forming a fluid tight (air and liquid impermeable) seal along the overlapping portions.

The material comprising the capsule portions is not particularly limited. Preferably, the capsule portions comprise material possessing a low oxygen transmission rate. For example, it is preferred the capsule portions comprise a material having an oxygen transmission rate (as measured by ASTM D3985) of less than about 165 cm³/m²/day for 100 μm, more preferably less than about 4 cm³/m²/day for 100 μm, and most preferably less than about 1 cm³/m²/day for 100 μm. Exemplary materials comprising the capsule portions include, but are not limited to, an ingestible material such as gelatin, hydroxypropyl methylcellulose, or starch. By way of specific example, the material may include gelatin having an oxygen transmission rate of about 3.5 cm³/m²/day for 100 μm. The resulting capsules may include hard gelatin capsules or soft gelatin capsules having an oxygen transmission rate of up to about 0.04 cm³/capsule/day (ASTM D3985 at 27° C. and rel. humidity of 50%).

In addition, opaque capsules are highly preferred. This can be achieved by adding pigment such as titanium dioxide to the capsule material formulation. Titanium dioxide is inert and possesses a high molecular weight, which prevents it from being absorbed into blood circulation when ingested. Opaque capsules function to prevent the degradation of the resveratrol-containing composition by light degenerative processes such as photooxidation. A commercially available, opaque capsule having low oxygen permeability is available from Capsugel (Greenwood, S.C.—www.capsugel.com), sold under the trade name Licaps®.

The system used to encapsulate the composition including biologically active small molecules material must create a fluid-tight (air and liquid impermeable) seal around capsule portions. A particularly preferred encapsulation system and process is disclosed in WO 01/08631 A1, incorporated herein by reference in its entirety. In this system and associated process, a capsule head portion and a capsule body portion are placed in a filling chamber. The capsule body portion is filled with the desired dosage material, and the capsule portions are then telescopically joined such that the head portion partially overlaps the body portion. A sealing liquid including a solvent is applied in the gap formed between the overlapping sections, and the capsule is dried to remove the solvent and form a fluid-tight seal.

It is important to the invention that the encapsulation process occurs in a substantially oxygen-free environment. In addition, it is preferred the encapsulation process take place in a darkened (substantially light free) environment. As explained above, small molecules such as resveratrol lose their biological activity upon exposure to light and/or oxygen (due, e.g., to oxidation processes). Consequently, the composition containing small molecules should be mixed and/or encapsulated in a system including airtight and darkened mixing and filling chambers having a substantially oxygen-free environment. This can be achieved by using an enclosed system from which oxygen is removed. Oxygen may be removed using a vacuum, replacing the oxygen within the system with an inert gas flush, or a combination thereof. For example, the system can be purged of oxygen using a controlled nitrogen blanket. In addition, the system is kept substantially oxygen free through the use of a nitrogen flush during the encapsulation process. A nitrogen purge may also be used to remove oxygen from each individual capsule. Specifically, prior to sealing, a positive pressure can be applied to each capsule to replace any oxygen present within the capsule with nitrogen. Upon sealing, a nitrogen bubble remains within the capsule. A commercially available encapsulation system capable of filling capsules in a substantially oxygen-free and light-free environment is available from Capsugel (Greenwood, S.C.—www.capsugel.com), sold under the trade name CPS 1000 Capsule Filling Machine.

The above process produces an encapsulated composition suitable as an orally ingestible dosage of biologically active small molecules (e.g., resveratrol). The composition including the biologically active small molecules may include up to 100% by weight small molecule material. In addition, the composition may contain additives to stabilize the biological activity of the small molecules, to improve their biological availability upon ingestion, and/or to improve their absorption and passage through biological barriers. For example, the composition may include one or more of (1) a chelating agent, (2) an antioxidant, and (3) an emulsifier.

A chelating agent (chelator) may be added to further preserve the biological activity of the small molecule material by preventing metal-induced oxidation. Oxygen, in the presence of metals such as iron, may be reduced to hydrogen peroxide by phenols. Hydrogen peroxide, in turn, may oxidize small molecules such as resveratrol and quercetin. Metals such as iron have special oxygen transfer properties, which, when combined with hydrogen peroxide, produce a more reactive and destructive form of iron, namely, Fe³⁺. In a Fenton reaction, an iron II (Fe²⁺) salt reacts with hydrogen peroxide to form an iron III (Fe³⁺) salt and a highly reactive hydroxyl radical.

Consequently, it is believed obstructing the Fenton reaction can block the oxidation of small molecules derived from a plant or synthetic source (e.g., resveratrol and quercetin). This can be achieved through the use of a chelating agent. For example, a metal chelator such as NDGA (nordihydroguaiaretic acid: 1,4-bis[3,4-dihydroxyphenyl]2,3dimethylbutane) functions to counter the oxidation of resveratrol by hydrogen peroxide. NDGA functions, in part, by converting the more reactive form of iron (Fe³⁺) to its less reactive form (Fe 2+). See Pinto et al., “Oxidation of Resveratrol Catalyzed by Soybean Lipoxygenase”, J. Agric. Food Chem., 51(6) (2003), 1653-1657; incorporated herein by reference in its entirety.

Phytic acid (also called inositol hexaphosphate) is an iron-binding molecule typically used as a food preservative due to its ability to block iron-driven oxidation, similar to the action of NDGA. Similarly, phytic acid functions as a metal chelator that minimizes, if not prevents, the occurrence of the Fenton reaction. See Graf et al. “Phytic Acid: A Natural Antioxidant”, J. Biol. Chem., August 1987, 262: 11647-11650; incorporated herein by reference in its entirety. Phytic Acid is a naturally derived material that comes from whole grains and seeds of plant sources, including corn, wheat, rice, soybean, sesame, and oat. The amount of chelating agent is not particularly limited, so long as it is sufficient to bind metals within the composition. By way of example, the chelating agent may be present in a range of about 0-25% by weight, more preferably in a range of about 5-15% by weight, and most preferably in a range of about 7-10% by weight.

In addition, an antioxidant may be added to the composition not only to provide additional biological activity to the composition, but also to prevent the degradation of the composition caused by oxidation. In biological systems, the normal processes of oxidation (plus a minor contribution from ionizing radiation) produce highly reactive free radicals. These can readily react with and damage other molecules. In some cases, the body uses this to fight infection. In other cases, the damage may continue to the body's own cells. Consequently, the presence of an antioxidant can prevent free radicals from damaging the small molecules. By way of example, the composition may include a phenolic antioxidant such as a flavonoid. By way of further example, the composition may include a flavanol compound such as quercetin. Quercetin, in addition to having antioxidant properties, is a small molecule plant polyphenol that exhibits enzymatic biological activity (including sirtuin enzyme activation) similar to that of resveratrol. Quercetin also functions to prohibit the sulfation of resveratrol once ingested. The amount of antioxidant in the composition is not particularly limited. By way of example, the antioxidant may be present in a range of about 0-50% by weight, more preferably in a range of about 15-35% by weight, and most preferably in a range of about 20-30% by weight.

An emulsifier may be added to the composition to enhance the bioavailability of the composition (i.e., to enhance the ability of the body to absorb and use the small molecules once ingested). By way of example, the emulsifier may comprise a phospholipid such as lecithin (phosphatidylcholine). The amount of emulsifier present in the composition is preferably in the range of about 0-50% by weight, more preferably in an amount of 15-45% by weight, and most preferably in a range of about 25%-40% by weight.

When one or more additives are present, the amount of material including small molecules is preferably in a range of about 1-70% by weight, more preferably in a range of about 5-30% by weight, and most preferably in an amount of about 10-20% by weight. By way of specific example, in a standard dietary capsule, the amount of resveratrol available for oral consumption is preferably in the range of about 3 to 70 mg. The additives may be combined with the material including small molecules at any time before the capsule is sealed. For example, the material including small molecules resveratrol material may be extracted, dried, mixed with an additive, and then encapsulated. Alternatively, the additives may be placed in the capsule before or after the material including small molecules is placed in a capsule.

Once the composition containing the small molecules is encapsulated, the resulting capsules may be packaged to prevent degradation of the small molecules in the event a capsule ruptures. For example, the capsules may be individually enclosed in a blister pack-including an airtight compartment. Additionally, if the capsules are stored loosely in an airtight, vacuum-sealed container (e.g., a bottle), a substantially oxygen-free environment can be maintained within the container by adding an oxygen absorbing packet capable of maintaining the amount of free oxygen within the bottle to less than about 100 parts per million or less. Preferably, any packaging used is flushed with nitrogen before sealed.

The present invention comprises a practical, economic method of maintaining the biological activity of components present in a composition containing small molecule plant polyphenols such as resveratrol by preventing the degradation of the components. Specifically, the inventive compositions and processes are believed to maintain the biological activity of the composition by preventing oxidation caused by oxidizing metals (i.e., metals naturally found in dietary supplement formulas, or metals provided in trace amounts as part of herbal extracts, or metals on surfaces of formulation, mixing and encapsulation machinery), which can trigger or accelerate the oxidation (spoilage) of the composition and destroy the biological activity of the components. This is further achieved by stabilizing the ingredient(s) in question against metal-induced oxidation through the addition of a chelating agent, and, as noted above, performing the handling and encapsulation of the dietary supplement in a substantially oxygen-free environment.

The small molecules present in the inventive encapsulated compositions maintain biological activity for substantial time periods after the normal life of the small molecules. For example, under ambient conditions, the half-life of resveratrol is approximately one day. In the manufacture and distribution of dietary supplements, however, it typically takes several weeks after encapsulation before the composition reaches a consumer. The embodiments of the instant invention form dietary supplements including small molecules that remain biological active upon reaching the consumer. In particular, it forms dietary supplements including resveratrol capable of enzymatic biological activity. Preferably, the small molecules remain biologically active for at least about four months after encapsulation, more preferably at least about eight months after encapsulation, and most preferably at least about one year or indefinitely.

Biologically active small molecules derived from a plant source or formed synthetically, as well as compositions containing biologically active small molecules, provide various health benefits. For example, biologically active small molecules are capable of enzymatic activity, activating human “longevity genes”. Studies have identified a class of regulatory genes that are shared by almost all living organisms. These genes function as a feedback system to enhance survival during times of stress, such as during drought or famine. Once activated, these longevity genes induce defensive changes at the cellular level, such as slowing metabolism and enhancing cellular respiration, to help the body adapt to an adverse environment. One particular stress, the restriction of calories provided to an organism, extends lifespan in numerous species by activating enzymes (or proteins) called sirtuins (a family of deacetylases).

Studies have shown that sirtuins (Silent Information Regulator enzymes) act as longevity genes due to their ability to control the rate of aging in organisms such as such as yeast, roundworms, and fruit flies. Specifically it has been shown that SIR2 (Silent Information Regulator gene 2) in yeast cells, which is homolgous to Sirtuin 1 in humans, becomes activated when under biological stress. In yeast, aging is directly linked to SIR2 activity. Overexpression of SIR2 increases DNA stability, increases silencing and suppression of rDNA recombination, speeds cellular repairs, and enhances mother-daughter cell replicative lifespan.

There are seven human sirtuins, which have been designated Sirtuin 1 through Sirtuin 7. Sirtuin 1 (SIRT1), which is located in the nucleus, is a human sirtuin having the greatest homology to SIR2. Human sirtuins appear to act as guardian enzymes that protect cells and enhance cellular survival. SIRT1, for example, has been shown to suppress the p53 enzyme system normally involved in suppressing tumor growth and instigating cell death (apoptosis). Inhibiting the activity of a tumor suppressor gene may not be readily deemed to be beneficial until it is recognized that SIRT1 inhibition prevents the cycle of premature aging and apoptosis normally. By suppressing p53 activity, SIRT1 prevents the cycle of premature aging and apoptosis normally induced when cellular DNA is damaged or stressed, thus giving cells enough time to repair any damage and prevent unnecessary cell death.

Studies, both in vitro and in vivo, have determined that small molecules such as resveratrol are capable of activating SIR2. In yeast, resveratrol decreases rDNA recombination and extends lifespan, similar to that which occurs during calorie restriction. Small molecules such as resveratrol, moreover, have been shown to activate SIRT1 in human cells, enhancing the survival rate of cells stressed by irradiation. Consequently, maintaining biological activity of small molecules enables the dosage present in, e.g., a dietary supplement including a composition formed and encapsulated using the above-described process, to activate longevity genes, namely the sirtuin enzymes SIR2 and SIRT1. In humans, the activation of the SIRT1 enzyme increases the survival rates of human cells, suppressing apoptosis.

The encapsulated compositions including small molecules are suitable for use in making dietary supplements, as well as prescription medications.

### EXAMPLE

Small molecules in the form of resveratrol were obtained via ethanol extraction from vitis vinifera and polygonum cuspidatum. The ethanol was removed, and the resulting extract comprised approximately 25% vinis vinifera skin resveratrol and 25% polygonum cuspidatum resveratrol, with the remainder comprising non-resveratrol, inert plant material. The biological activity of the resveratrol in the extract was confirmed using a SIRT1 Fluorescent Activity Assay/Drug Discovery Kit AK-555 (available from Biomol® Research Laboratories, Inc.; Plymouth Meeting, Pa.; www.biomol.com). The extract was kept in a nitrogen environment and added to a mixture including approximately 25% by weight quercetin; 33% by weight lecithin; and 9% phytic acid (in the form of rice bran extract). The remainder of the composition included approximately 33% by weight resveratrol extract. The resulting slurry was placed into a capsule-filling machine. Individual dosages were encapsulated in gelatin capsules tinted with titanium oxide (Licaps® capsules available from Capsugel; Greenwood, S.C.; www.capsugel.com). The dosages were encapsulated in a substantially oxygen-free environment using a capsule-filling machine continually flushed with nitrogen (the Capsugel CFS 1000 Capsule Filling and Sealing Machine, available from Capsugel; Greenwood, S.C.; www.capsugel.com). Each resulting capsule included at least 15 mg resveratrol, 100 mg lecithin, 75 mg quercetin, and 25 mg phytic acid. These capsule samples were stored under ambient conditions for approximately eight months. The samples were tested for biological activity by determining whether each sample could activate sirtuin enzymes and, in particular, whether the samples stimulated SIRT1 catalytic activity. The samples were tested four months and eight months after encapsulation. Tests were performed using a SIRT1 Fluorescent Activity Assay/Drug Discovery Kit AK-555 (available from Biomol® Research Laboratories, Inc.; Plymouth Meeting, Pa.; www.biomol.com). Upon testing, it was determined that the resveratrol contained within the samples was biologically active, stimulating SIRT1 activity, producing up to about an eight-fold stimulation in enzymatic activity compared to when no resveratrol is present. Similarly, the biological activity of the quercetin was tested, and it was determined that the encapsulated quercetin maintained biological activity (i.e., the ability to stimulate SIRT1 activity compared to when no quercetin is present).

The invention is comprised of multiple methods of manufacturing and preserving maximum biological activity (including having the ability to activate sirtuin enzymes) and the structural form of plant polyphenols and other dietary supplement ingredients against degradation (via, e.g., oxidation), including but not limited to the manufacture of raw material and its placement into capsules. The above-described method economically produces an encapsulated composition including concentrated resveratrol having properties similar to that made available in research-grade resveratrol or resveratrol as it exists in a sealed wine bottle. The present invention is useful in manufacturing encapsulated formulations that can be ingested as a dietary supplement.

While the invention has been described in detail and with reference to specific embodiments thereof, it will be apparent to one skilled in the art that various changes and modifications can be made therein without departing from the spirit and scope thereof. For example, the capsule may be of any shape and size, and may be made breakable to enable the removal of the composition from the capsule. The capsule material, moreover, may comprise any material having a low oxygen transmission rate. The compositions including small molecules, furthermore, may exist as a dried powder, a liquid suspension, a gel, or a slurry. In addition, the formulations may include other additives, fillers, etc. suitable for dietary supplement formulations, so long as the additives do not degrade the biological activity or the bioavailabilty of the material including small molecules. In addition, other small molecule polyphenols capable of enzymatic activity (including stimulating the catalytic activity of sirtuins) may be encapsulated using the above-described process, including but not limited to quercetin, fisetin, butein, piceatonol, and isoliquiritigenin. Thus, it is intended that the present invention covers the modifications and variations of this invention that come within the scope of the appended claims and their equivalents.

1. A process of encapsulating a composition including the steps of:
(a) deriving material including biologically active small molecules; and
(b) encapsulating the biologically active small molecules in a substantially oxygen-free environment.

2. The process of claim 1, wherein the biologically active small molecules comprise resveratrol.

3. The process of claim 2 further including (c) combining the resveratrol with at least one of a chelating agent, an antioxidant, and an emulsifier.

4. The process of claim 2, wherein step (a) includes:
(a. 1) using a solvent to extract the resveratrol, wherein said solvent includes aqueous solvents, organic solvents, and mixtures thereof.

5. The process of claim 2, wherein step (b) includes:
(b. 1) receiving a capsule including a head portion and a body portion into an airtight filling chamber;
(b.2) at least partially filling the body portion with a composition including the biologically active resveratrol;
(b.3) positioning the head portion over the body portion such that the portions at least partially overlap; and
(b.4) forming a fluid tight seal along the overlapping portions.

6. The process of claim 5, wherein the capsule portions comprise a material having an oxygen transmission rate (as measured by ASTM D3985) of less than about 165 cm³/m²/day for 100 μm,

7. The process of claim 5, wherein the capsule portions comprise a material having an oxygen transmission rate (as measured by ASTM D3985) of less than about 4 cm³/m²/day for 100 μm.

8. The process of claim 5, wherein the capsule portions comprise a material having an oxygen transmission rate (as measured by ASTM D3985) of less than about 1 cm³/m²/day for 100 μm.

9. The process of claim 5, wherein the capsule portions comprise at least one of gelatin, hydroxypropyl methylcellulose, and starch.

10. The process of claim 5, wherein step (b) further includes
(b.5) forming a nitrogen blanket within said filling chamber to form the substantially oxygen-free environment.

11. The process of claim 4, wherein step (b) further includes
(b.6) forming a nitrogen bubble within the capsule prior to the sealing step.

12. The process of claim 2, wherein step (a) further includes:
(a. 1) deriving said resveratrol from a natural source selected from the group consisting of Vinis Vinifera and Polygonum.

13. An encapsulated composition including biologically active small molecules, a chelating agent, an antioxidant, and an emulsifier, wherein the small molecules were encapsulated in a substantially oxygen-free environment.

14. The encapsulated composition of claim 13, wherein said biologically active small molecules comprise resveratrol.

15. The encapsulated composition of claim 14, wherein:
said chelating agent is present in an amount of about 5-15% by weight,
said antioxidant is present in an amount of about 15-35% by weight,
said emulsifier is present in an amount of about 15-45% by weight, and
said resveratrol is present in an amount of about 5-30% by weight.

16. The encapsulated composition of claim 14, wherein:
said chelating agent is present in an amount of about 7-10% by weight,
said antioxidant is present in an amount of about 20-30% by weight,
said emulsifier is present in an amount of about 25-40% by weight, and
said resveratrol is present in an amount of about 10-20% by weight.

17. The encapsulated composition of claim 14, wherein said chelating agent comprises a compound capable of blocking metal-induced oxidation of resveratrol.

18. The encapsulated composition of claim 17, wherein said chelating agent comprises at least one of phytic acid and NDGA.

19. The encapsulated composition of claim 14, wherein said antioxidant comprises a phenolic antioxidant.

20. The encapsulated composition of claim 19, wherein said phenolic antioxidant comprises quercetin.

21. The encapsulated composition of claim 14, wherein said emulsifier comprises lecithin (phosphatidylcholine).

22. The encapsulated composition of claim 14, wherein said resveratrol is derived from a natural source.

23. The encapsulated composition of claim 14, wherein said biological activity includes activating Sirtuin 1 enzymes.

24. A dietary supplement produced by the process of
(a) deriving biologically active resveratrol material;
(b) receiving a capsule including a head portion and a body portion into a filling chamber comprising a substantially oxygen-free environment;
(c) at least partially filling the body portion with the biologically active resveratrol material;
(d) positioning the head portion over the body portion such that the portions at least partially overlap; and
(e) forming a fluid tight seal along the overlapping portions.

25. A dietary supplement including an encapsulated composition comprising resveratrol in its biologically active form, wherein said resveratrol maintains biological activity for at least four months after encapsulation.

26. The dietary supplement of claim 25, wherein said biological activity includes stimulating Sirtuin 1 enzyme activity.

27. An encapsulated composition including biologically active small molecules and at least one of a chelating agent, an antioxidant, and an emulsifier, wherein said small molecules are capable of passing through a cell wall, entering the cell nucleus and altering gene-controlled enzymes located within the nucleus.

28. The encapsulated composition of claim 27, wherein said gene-controlled enzymes are Sirtuin 1 enzymes.
